(19) United States
(12) Patent Application Publication
(10) Pub. No.: US 2005/0149144 A1
(43) Pub. Date: Jul. 7, 2005

(54) Stimulation of central nervous system

(76) Inventor: David Siever, Edmonton (CA)

Correspondence Address: KIRBY EADES GALE BAKER, BOX 3432, STATION D, OTTAWA, ON, K1P 6N9, CA

(21) Appl. No.: 10/748,154

(22) Filed: Dec. 31, 2003

(51) Int. Cl. A61N 1/18

(52) U.S. Cl. 607/45

(57) ABSTRACT

The present invention provides a method of stimulating the central nervous system and brain waves of a human subject by stimulating a beta frequency in the left brain hemisphere while simultaneously stimulating a low beta frequency in the right brain hemisphere and subsequently stimulating the left and the right brain hemispheres at an alpha frequency. The present invention also provides a process for suppressing aberrant brain wave frequencies, by stimulating the brain at approximately twice the aberrant brain wave frequency. The present invention further provides a process for dissociating a subject by stimulating a left brain hemisphere at a frequency that differs by 0.1 to 3 Hz from a frequency at which right brain hemisphere is stimulated. The present invention also provides a method of pacing breathing in a subject to a predetermined rate comprising exposing the subject to an auditory cue and simultaneously exposing the subject to various frequencies and combinations of stimulation.

### FIELD OF THE INVENTION

The present invention relates to methods of stimulating the central nervous system using photic/light and audio based stimulation.

### BACKGROUND OF THE INVENTION

It is widely supported in the field of psychology and learning that the left hemisphere of the brain is the source of logical reasoning, auditory and speech processing, and rote functioning of the human consciousness. Conversely, the right brain hemisphere is the source of spatial, artistic, creative and imaginative functioning within the brain.

Various methods have been developed for use in stimulating and patterning these brain functions. For example, U.S. Pat. No. 6,443,977, related to U.S. Pat. No. 6,299,632 and U.S. published application 2002/0 198577, teaches a method of treating a number of neurological disorders by using light and/or sound to stimulate the non-dominant eye and the non-dominant cerebral hemisphere. Another example is U.S. published application 2001/0056293, which teaches a method of using light to stimulate or regulate various systems in the body and to treat a number of disorders and deficits which could be classified as neurological or psychological disorders.

A problem with these stimulation systems is that they stimulate the whole eye and do not account for the distinct visual fields that exist in each eye. It has been found that the left visual field of both eyes elicit responses from the right visual cortex of the brain; and the right visual field of both eyes elicit responses from the left visual cortex of the brain. This visual field concept permits better control over left and right brain stimulation.

Consequently, there is a need for a photic stimulating method for stimulating the left and right visual fields of each eye independently of each other. There is also a need for a method of synchronizing both photic and auditory stimulation.

### SUMMARY OF THE INVENTION

Various exemplary embodiments of the present invention relate to methods of stimulating the central nervous system and brain waves of a human subject having left and right eyes and left and right visual fields within each eye, by providing pulsating light signals to the visual fields of each eye of the subject; and varying frequency and intensity of the light signals.

In accordance with various aspects of the present invention there are provided methods for stimulating the central nervous system and brain waves of a human subject by stimulating a beta frequency in a range of from 15 to 20 Hz in the left brain hemisphere for a first period of time while simultaneously stimulating a low beta frequency in a range of from 12 to 15 Hz in the right brain hemisphere for the same first period of time and subsequently stimulating the left and the right brain hemispheres at an alpha frequency in a range of from 8 to 12 Hz also for a second period of time. These steps are repeated for up to 6 cycles to complete a session.

The present invention also provides a process for suppressing aberrant brain wave frequencies in a brain of a subject, comprising stimulating the brain at a frequency that is approximately twice the frequency of the aberrant brain wave frequency.

The present invention further provides a process for dissociating a subject from self awareness comprising stimulating a left brain hemisphere at a first frequency and simultaneously stimulating a right brain hemisphere at a second frequency, wherein the first frequency differs from the second frequency by 0.1 Hz to 3 Hz.

The present invention additionally provides a process for reducing depression in a subject, comprising stimulating alpha frequencies in a range of from 8 to 12 Hz in the right brain hemisphere and stimulating beta frequencies in a range of from 15 to 20 Hz in the left brain hemisphere.

The present invention also provides a method of pacing breathing in a subject to a predetermined breathing rate in the range of from 5 to 7 breath cycles per minute comprising exposing the subject to an auditory cue to pace breathing while simultaneously applying various frequencies or combinations of stimulation.

### BRIEF DESCRIPTION OF THE DRAWINGS

FIG. 1 is a top plan view of a subject's head, showing a typical placement of electrodes for realizing the method of the present invention;

FIG. 2 is a graph of alpha brain wave activity at different electrode locations for a subject whose eyes are closed (EC);

FIG. 3 is a graph of alpha brain wave activity at different electrode locations for a subject who is reading;

FIG. 4 is a graph of alpha brain wave activity for a normal subject during four (eyes-closed, eyes-open, reading, and math) tasks;

FIG. 5 is a graph of beta brain wave activity for a normal subject during four tasks;

FIG. 6 is a graph of alpha brain wave activity for a subject suffering from attention deficit disorder (ADD) during three tasks;

FIG. 7 shows three quantitative electroencephalograms (QEEG) of a subject suffering from ADD, illustrating alpha brain wave activity before and after treatment with methods of the present invention;

FIG. 8 is a graph of alpha brain wave activity for a subject suffering from ADD, before treatment with low beta/SMR and alpha stimulation;

FIG. 9 is a graph of alpha brain wave activity for a subject suffering from ADD, after treatment with low beta/SMR and alpha stimulation;

FIG. 10(a) is a QEEG of alpha brain activity of a subject suffering from depression and anxiety, before treatment with alpha/beta stimulation;

FIG. 10(b) is a QEEG of alpha brain activity of a subject suffering from depression and anxiety, after treatment with alpha/beta stimulation;

FIG. 11(a) is a QEEG of beta brain activity of a subject suffering from depression and anxiety, before treatment with alpha/beta stimulation. and

FIG. 11(b) is a QEEG of beta brain activity of a subject suffering from depression and anxiety, after treatment with alpha/beta stimulation.

### DETAILED DESCRIPTION

The processes of the various embodiments of the present invention can be used with a photic stimulation device such as, for example, the stimulator of U.S. Pat. No. 5,709,645, incorporated herein by reference. In general, the photic device includes an eye mask with independent left and right eye pieces and means of fitting the eye mask over the subject's eyes. Each eyepiece contains a dedicated light-producing assembly having two independent sets of light sources, one for each of the left and right visual fields of each eye. Each of the light sources is independently operable to pulse light into the corresponding visual field of each eye, thereby stimulating that particular visual field. An optional blocker may be placed between the sets of light sources in each light-producing assembly, to prevent light sources from illuminating more than their associated visual field.

In an exemplary embodiment of the present invention an auditory stimulation can also be applied to a subject's audio sense using a set of headphones that receive pulses of sounds in the form of pulsed tones at specific frequencies to influence brain wave activity in the contra-lateral hemisphere of the brain. Two discrete brain wave frequencies may be generated within the brain simultaneously. Audio stimulation can optionally be used in conjunction with visual stimulation, in which case the method would involve pulsing the tones at the same frequency as the lights, for each side of the subject's brain.

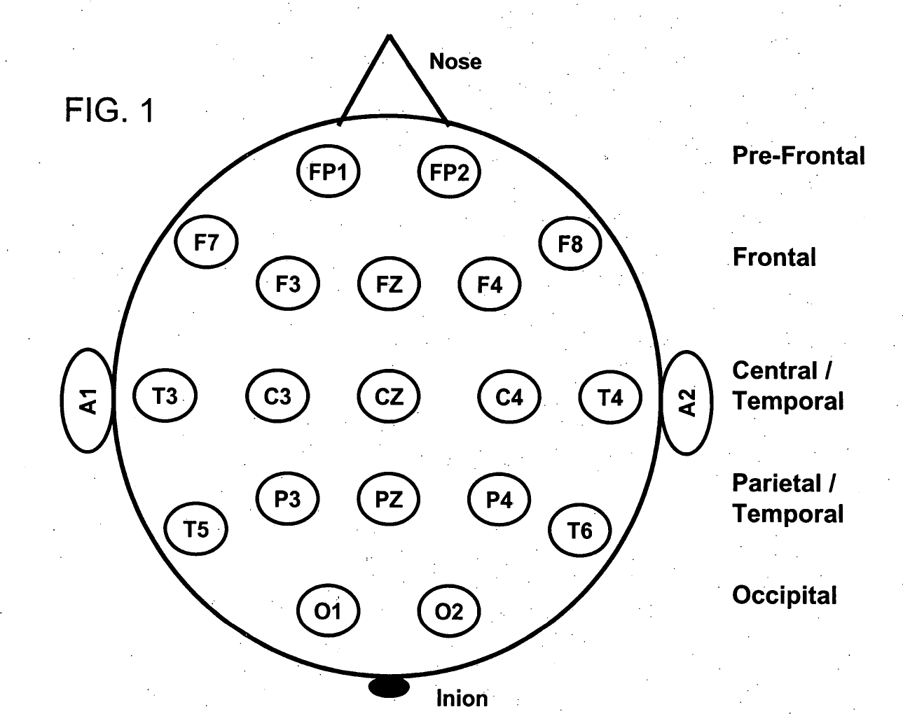

Brain wave activity before, during and after treatment is measured by placing electrodes on the head of a subject. Any typical electrode placement configuration known in the art can be used for reading and recording a subject's brain wave activity, both during stimulation and otherwise. FIG. 1 shows one example of such a configuration, namely a 10-20 configuration.

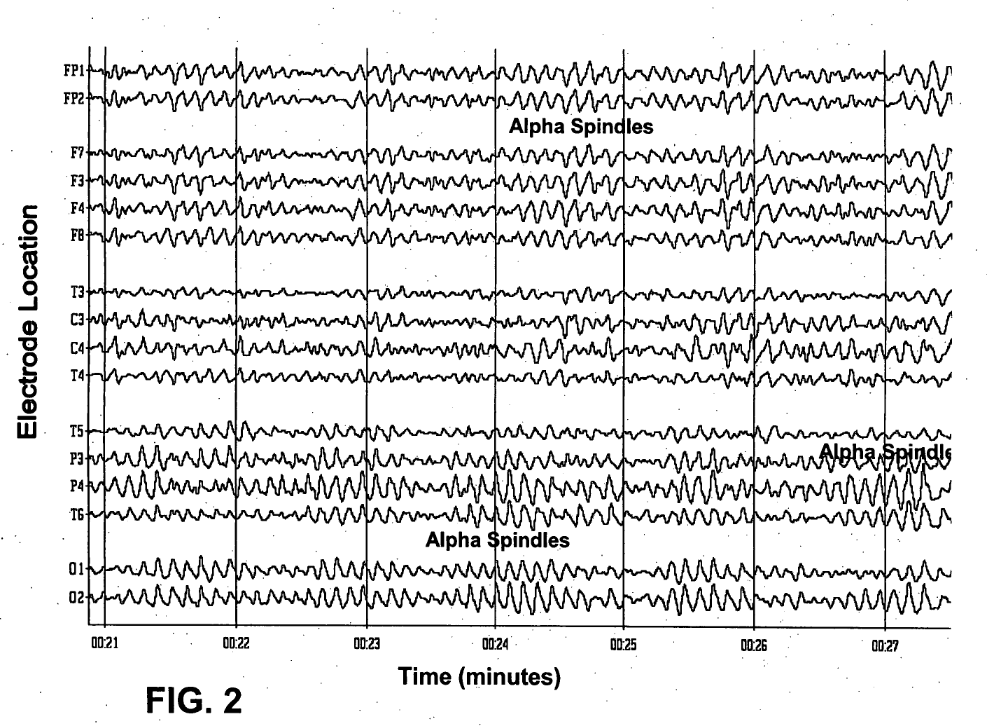

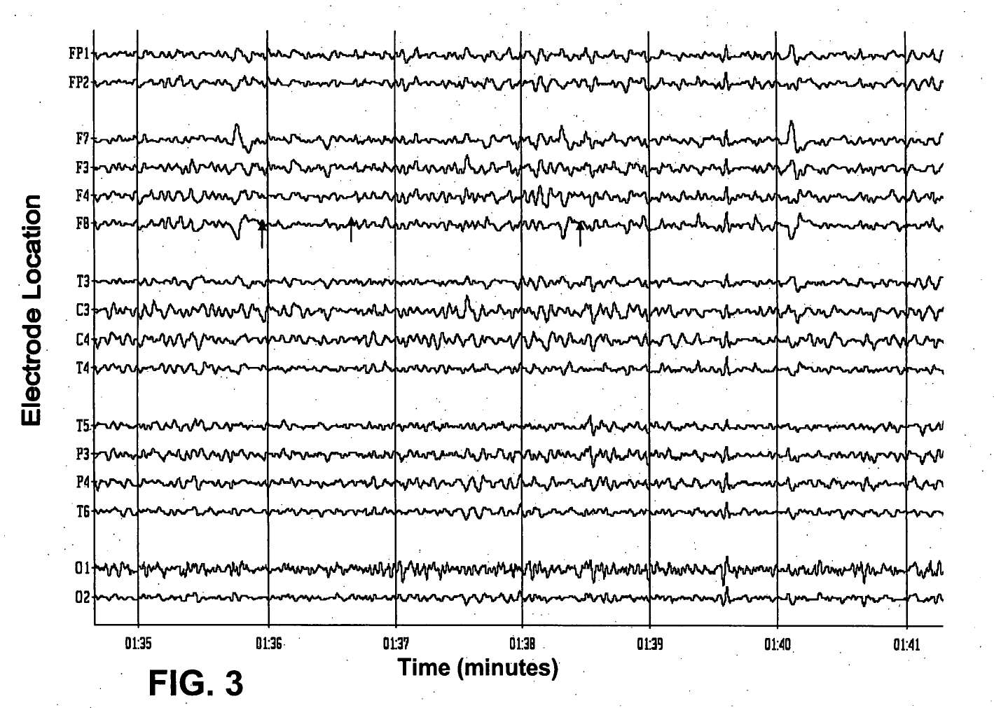

FIGS. 2 and 3 show the alpha brain wave activities at different electrode locations for a subject with eyes closed and reading, respectively. Typically, when a subjects eyes are open, and even more so if the subject is engaged in a task such as reading, alpha activity tends to become suppressed and beta brain waves begin to dominate. This trend can be seen in the relatively smaller amplitude of waves in FIG. 3 compared to FIG. 2. The arrow in FIG. 3 indicates a moment of higher amplitude activity that coincides with movement of the eyes across a page while reading. This activity occurs at electrode locations F7 and F8, which are seen in FIG. 1 to be closest to the eyes, and thus recording most of the eye movements.

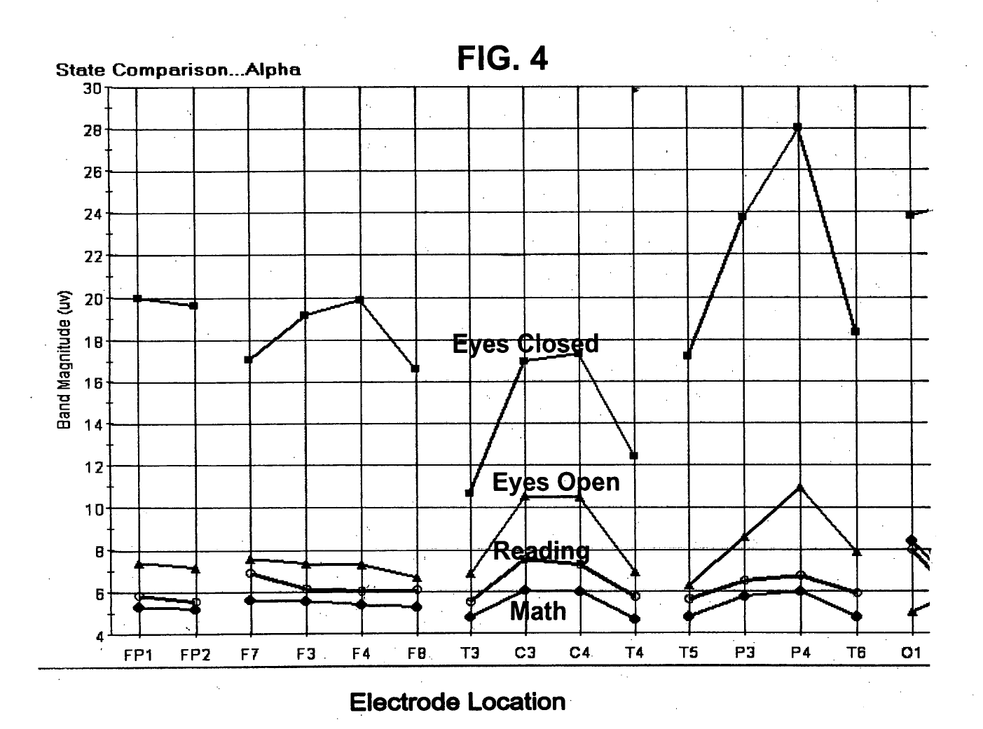

FIG. 4 shows the alpha brain wave activity, measured in micro-volts (uV), of a normal subject during four types of tasks, namely Eyes Closed (subject is awake, but with eyes closed), Eyes Open (subject is awake and idle, with eyes open), Reading (subject is engaged in reading) and Math (subject is engaged in mathematical computations). Alpha waves, which dominate during idle periods, are at their highest magnitude during Eyes Closed, as is clearly shown in FIG. 4. A brain is stimulated during mathematical tasks. In particular, alpha waves become suppressed and lower magnitude, higher frequency (beta) wave types tend to dominate.

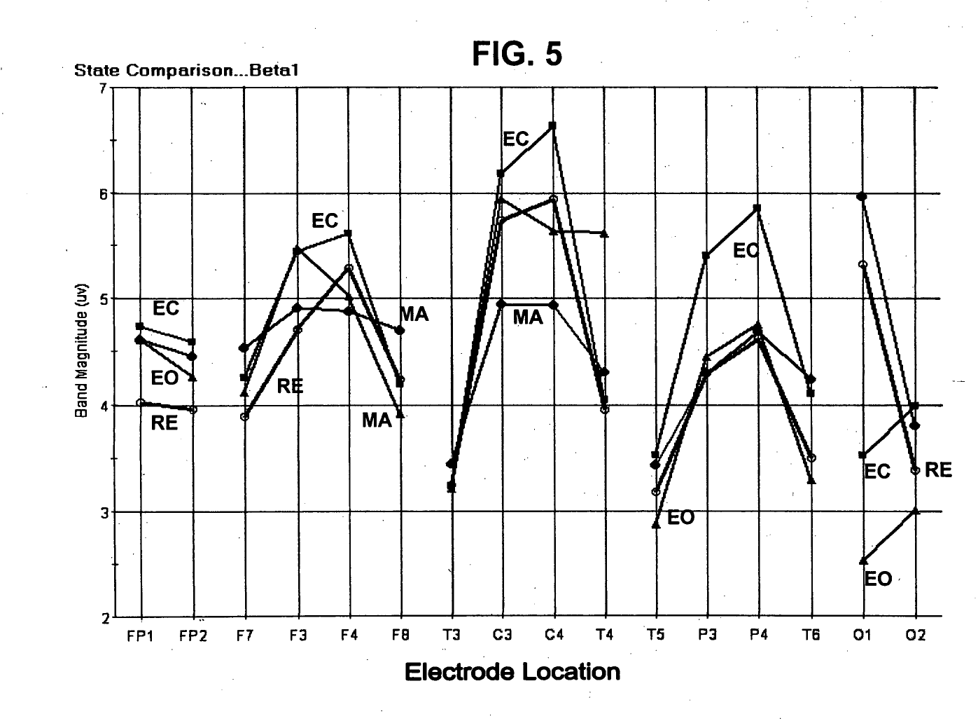

FIG. 5 shows beta brain wave activities for a normal subject during the same tasks as in FIG. 4. It can be seen from FIG. 5 that the magnitude of beta waves is much smaller than that of alpha waves, and that beta wave activities do not vary as much from task to task.

By exposing the subject to two discrete light frequencies, one for each side of the subjects eyes, two discrete brain wave frequencies can be simultaneously generated within the brain, one in each hemisphere (i.e., stimulation of the central nervous system using light pulses of various frequencies). Such brain wave generation can reduce or suppress aberrant brain wave frequencies that cause impulsiveness and hyperactivity while improving attention, mental functioning, reasoning ability, reaction times, reading speed, comprehension and retention. The process has also produced benefits in people with suffering from problems related to attention deficit disorder (ADD) and brain injury.

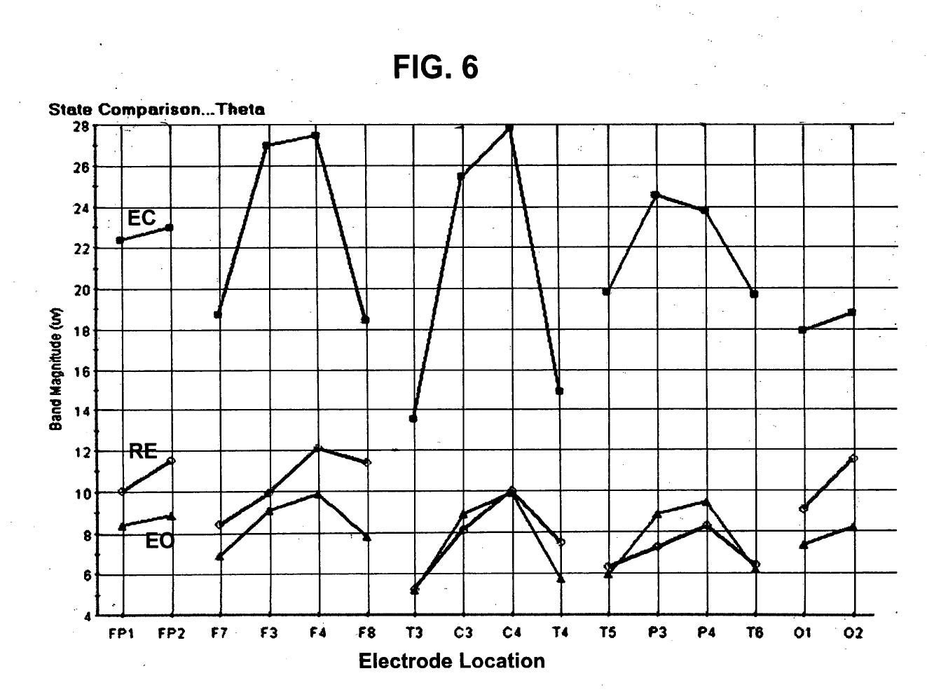

FIG. 6 illustrates the alpha brain wave activity of a subject suffering from attention deficit disorder (ADD), at three tasks; Eyes Closed (EC), Eyes Open (EO) and Reading (RE). FIG. 6 illustrates significantly higher alpha brain wave activities than seen in FIG. 4 for a normal subject. Also, it is seen that alpha activity is actually higher for reading than for eyes open, which is the opposite of alpha activity in normal subjects. This inversion in alpha, and sometimes theta, activity is commonly understood to be due to the subject “fogging up” or experiencing a mental block as he or she struggles to read. This is a common symptom in patients suffering from ADD.

In one embodiment, the process involves stimulating frequencies of from 15 to 20 Hz (beta frequencies) in the left hemisphere of the brain while simultaneously stimulating frequencies in the range of from 12 to 15 Hz (low beta frequencies) in the right hemisphere for a period of time. This step is followed by stimulating both hemispheres of the brain at frequencies of from 8 to 12 Hz (alpha frequencies) for a second period of time. The first period of time can be approximately similar to or different from the second period of time. The steps can be repeated for up to 6 cycles to complete a session. Preferably, the first and the second periods of time range between 2 and 10 minutes. This method can be used on a hyperactive child by using fairly sudden shifts of about 30 seconds between the beta/low beta and alpha frequencies. Sudden shifts enhance dissociation of the child and help to keep the child engaged in the session and prevent distraction. Alternately, slow shifts done in 0.1 Hz increments over a few minutes or more between beta/low-beta frequencies and alpha frequencies can be used for senior citizens who may feel uncomfortable or nauseas if subjected to a “sudden shift” or rapid transition approach.

In another embodiment, the process involves providing stimulation at approximately twice the frequency of an aberrant brain wave frequency as a method to suppress the aberrant frequency. For example, people suffering from seasonal affective disorder (SAD) generally produce long streams of 10 Hz alpha frequency waves, associated with mental fog, depression, lethargy and carbohydrate cravings. By stimulating the brain at a frequency of 20 Hz, these symptoms can be alleviated. In a further example, people suffering from fibromyalgia syndrome (FMS), in which the symptoms include musculoskeletal pain and fatigue, often exhibit excessive brain wave activity at frequencies of from 7 to 9 Hz. In this case, stimulating the brain at a frequency of from 14 to 18 Hz, can suppress the lower brain wave frequencies and help alleviate their symptoms.

Various embodiments of the present invention can also be used in relieving depression and depression related symptoms. In this case, the process involves stimulating alpha frequencies in a range of from 8 to 12 Hz in the right hemisphere and beta frequencies in a range of from 15 to 20 Hz in the left hemisphere of the brain.

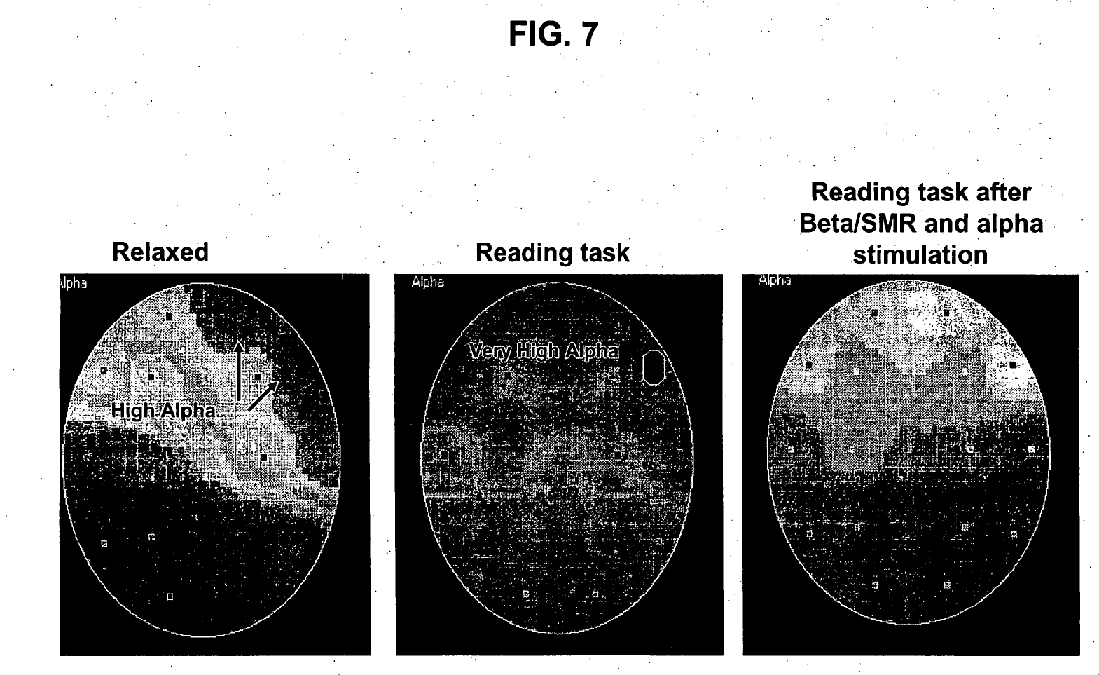

Various embodiments of the present invention can also serve to suppress detrimentally high alpha brain wave levels to allow the subject to perform cognitive tasks at hand and to improve concentration. FIG. 7 shows the results the alpha brain wave levels in a subject at rest, during a reading task, and then during a reading task after being exposed to treatment with a process of the present invention. The subject is treated to beta stimulation in the left hemisphere and low beta stimulation or sensory motor rhythm (SMR) stimulation in the right hemisphere, cycled with periods of alpha stimulation in both hemispheres. Sensory motor rhythms are located in the sensory motor cortex of the brain, located approximately across the top of the head from the tips of the ears. Sensory motor rhythm is represented by brainwave activity between 13 and 15 Hz, similar to that of beta waves. Sensory motor rhythm governs body sensations and voluntary movement. In FIG. 7, dark patches represent alpha activity, whereas light patches indicate normal brain wave activities.

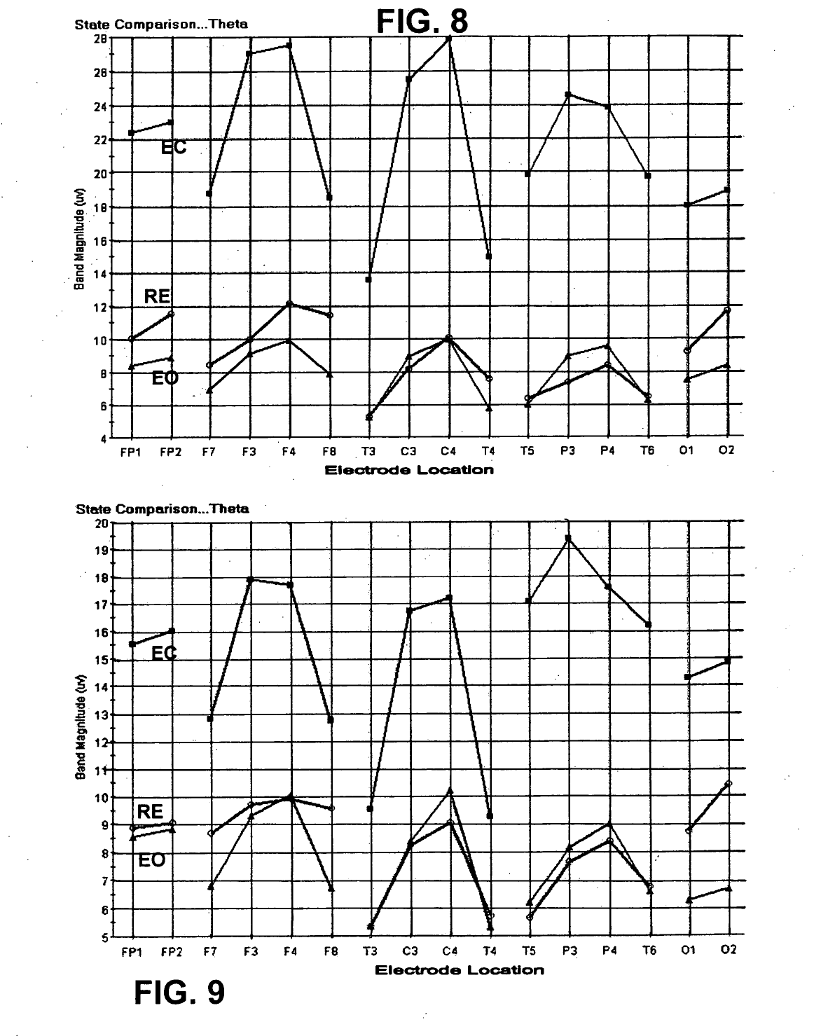

FIGS. 8 and 9 respectively show the alpha brain wave activities for a 22 year old subject suffering from ADD, before and after treatment with low beta/SMR and alpha stimulation. In comparing FIG. 9 to FIG. 8, it can be seen that alpha brain waves are significantly suppressed after the subject is exposed to the low beta/SMR and alpha stimulation for all three types of tasks. As well the inversion effect during reading is lessened and the alpha brain wave levels for reading are also decreased from 11 uV to 9 uV.

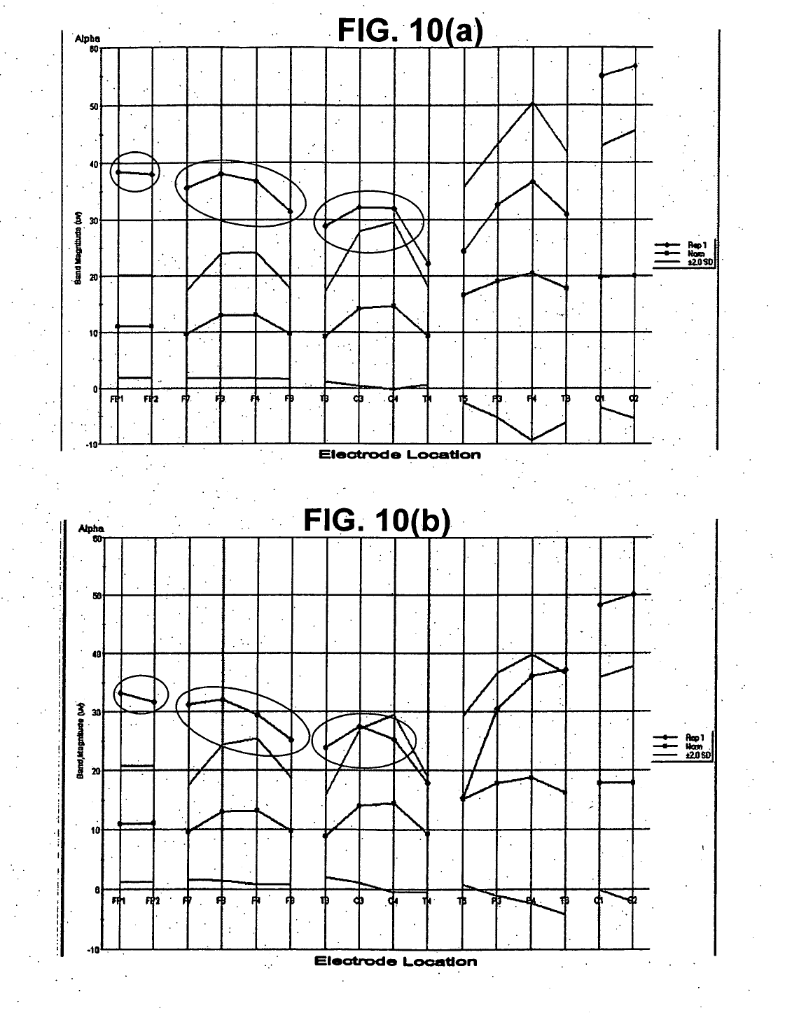

FIGS. 10(a) and (b) show QEEGs of alpha brain activity of a subject suffering from depression and anxiety, before and after treatment with alpha/beta stimulation. Before stimulation, as seen FIG. 10(a), the pre-frontal, frontal and central areas of the brain show high alpha activity, a common indicator of depression. By contrast, FIG. 10(b) shows greatly reduced alpha activity in the same brain areas, after treatment with alpha/beta frequency stimulation.

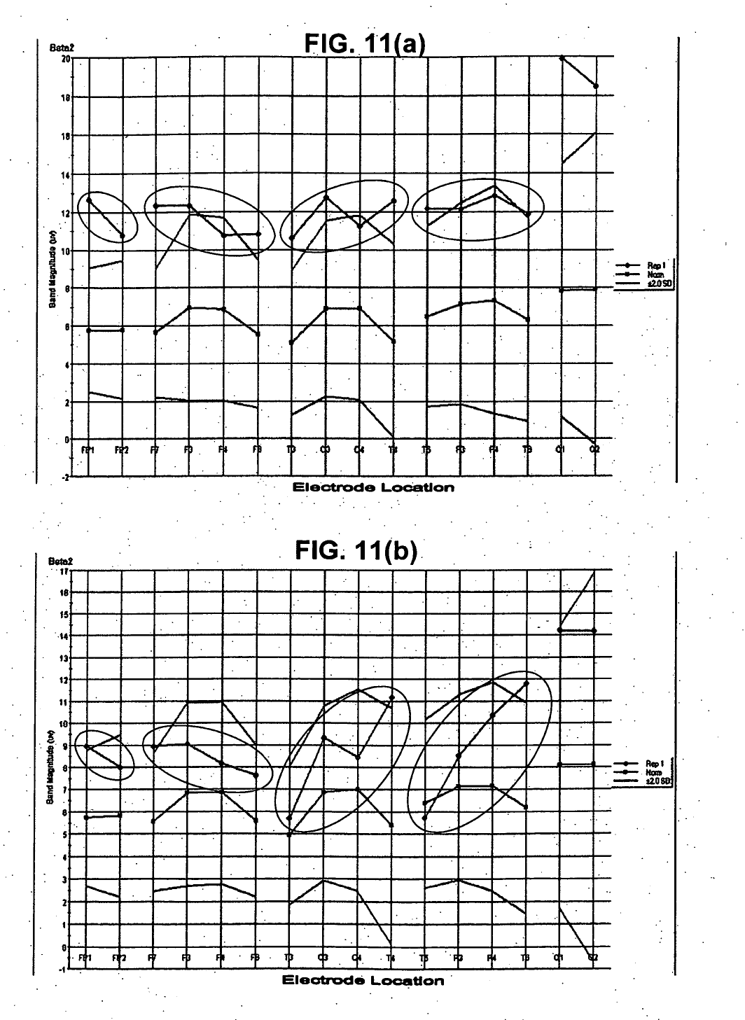

FIGS. 11(a) and (b) show QEEGs of beta brain activity of a subject also suffering from depression and anxiety, before and after treatment with alpha/beta stimulation. Beta brain wave activity is generally considered to correlate to the anxiety component associated with depression. Before stimulation, as seen FIG. 11(a), the pre-frontal, frontal, central, temporal parietal and occipital areas of the brain all show high beta activity. By contrast, FIG. 11(b) shows greatly reduced beta activity in the same brain areas, after treatment with alpha/beta frequency stimulation.

In a further embodiment, a process of stimulating the two hemispheres of the brain with two dissimilar frequencies is performed, as described above, wherein the two frequencies are within, for example, 0.1 and 3 Hz of each other. The use of two close yet different frequencies, hereinafter referred to as dissociation frequencies, has the effect of dissociating the subject from awareness of body and mind. This dissociation is similar to that experienced during, for example, hypnosis or meditation. The process is found to be particularly useful in reducing symptoms of anxiety, panic or depression. Applying dissociation frequencies to the hemispheres of the brain functions to block fearful, worrying or destructive thoughts and relax and stabilize the subject. This process is also effective for improving sleep as it reduces mental chatter, caused by anxiety and daily events that interfere with sleep.

The processes described above can also be combined into treatment regimes for the treatment of insomnia. The following examples illustrate some treatment regimes that combine various embodiments of the present invention.

### EXAMPLE 1

Dissociation frequencies are applied to the subject and alternated with low beta frequency stimulation in the range of from 12 to 15 Hz to improve sleep in those suffering from insomnia due to mental chatter and anxiety.

### EXAMPLE 2

Dissociation frequencies are applied to the subject and alternated with low alpha or theta frequency stimulation in the range of from 5 to 9 Hz to improve sleep in those suffering from insomnia due to physical tension, mental chatter and anxiety.

### EXAMPLE 3

Dissociation frequencies are applied to the subject and alternated with delta frequency stimulation in the range of from 0 to 4 Hz to improve sleep in those suffering from insomnia due to somatic and chronic pain resulting from injury or fibromyalgia syndrome (FMS).

### EXAMPLE 4

Because low alpha and theta stimulation reduces physical tension more quickly than it reduces generalized anxiety, dissociation frequencies are applied to the subject and alternated with low alpha or theta frequency stimulation in the range of from 5 to 9 Hz, followed by dissociation frequencies alternated with low beta frequency stimulation in the range of from 12 to 15 Hz to relax the body. The entire process can extend from three to 20 weeks.

The treatments described in examples 1 to 3 above have also been found useful in improving the performance of athletes. Athletes who often travel for sporting events may suffer from insomnia due to constant changes in locale, physical pain from exertion or injury or anxiety and mental chatter resulting from the pressure to excel in competitions, or the natural excitement and euphoria that often comes after intense sporting matches. Business executives with overwhelming agendas, students under academic pressure and people with anxiety and/or tension-induced pain have also been found to benefit. The treatment regimes of examples 1 to 3 can be used to relieve these symptoms and improve sleep.

Embodiments of the present invention also provide a method to pace the breathing of a subject suffering from anxiety, panic, depression or post traumatic stress disorder, so that the subject is breathing at roughly 5 to 7 breathe cycles per minute. This process can utilize synthesized heartbeat sounds or other auditory cues to regulate or pace breathing, simultaneously with various stimulation frequencies or combinations thereof to relax the subject. The auditory cues may be provided at 2 times or 4 times the breathing rate. A cue at 2 times the breathing rate would provide one prompt for inhale and one prompt for exhale. In this case, twelve prompts could be used to achieve a breathing rate of six breaths per minute. A cue at 4 times the breathing rate would provide two prompts for inhale and two prompts for exhale, totalling 24 prompts per minute for six breath cycles per minute. The method can be used to develop a conditioned response of regulated breathing during stressful or traumatic situations.

Although the methods of the present invention have been described in relation to human subjects, similar effects on brain wave activity have been found when using low-alpha (6-10 Hz) on mammal subjects to induce a calming effect. Therefore the scope of the present application is not limited to use only with humans.

1. A photic stimulation process comprising:
a. stimulating a beta frequency in a range of from 15 to 20 Hz in a left brain hemisphere for a first period of time;
b. simultaneously stimulating a low beta frequency in a range of from 12 to 15 Hz in the right brain hemisphere for the first period of time;
c. stimulating the left brain hemisphere and the right brain hemisphere at an alpha frequency in a range of from 8 to 12 Hz for a second period of time; and
d. repeating steps (a), (b) and (c) from 1 to 6 times.

2. A process according to claim 1 wherein the first period of time is approximately equal to the second period of time.

3. A process according to claim 1 wherein the first and the second periods of time range from 2 to 10 minutes.

4. A process according to claim 1 wherein a shift from steps (a) and (b) to step (c) is sudden and the shift takes about 30 seconds.

5. A process according to claim 1 wherein a shift from steps (a) and (b) to step (c) is ramped in 0.1 Hz increments and the shift takes at least three minutes.

6. A process comprising: (a) obtaining a frequency of aberrant brain waves in a brain; and (b) stimulating the brain at a frequency that is approximately twice the frequency of the aberrant brain waves to suppress the aberrant brain waves.

7. A process according to claim 6 wherein the frequency of stimulation in step (b) is approximately 20 Hz based on the aberrant brain wave frequency in the brain being approximately 10 Hz for seasonal affective disorder (SAD).

8. A process according to claim 6 wherein the frequency of stimulation in step (b) is between approximately 14 to 18 Hz based on the aberrant brain wave frequency in the brain being approximately 7 to 9 Hz for fibromyalgia syndrome (FMS).

9. A process for dissociating a subject from self awareness comprising: (a) stimulating a left brain hemisphere at a first frequency using photic stimulation and (b) simultaneously stimulating a right brain hemisphere at a second frequency using photic stimulation, wherein the first frequency differs from the second frequency by between approximately 0.1 Hz and 3 Hz.

10. A process according to claim 9, wherein the process is alternated with stimulating the left brain hemisphere and the right brain hemisphere at a low beta frequency in a range of from 12 to 15 Hz.

11. A process according to claim 9, wherein the process is alternated with stimulating the left brain hemisphere and the right brain hemisphere at low-alpha or theta frequencies in a range of from 5 to 9 Hz.

12. A process according to claim 9, wherein the process is alternated with stimulating the left brain hemisphere and the right brain hemisphere at a delta frequency in a range of from 0 to 4 Hz.

13. The process of claim 11, followed by the process of claim 10, conducted for from 3 to 20 weeks.

14. A photic stimulation process comprising: (a) stimulating alpha frequencies in a range of from 8 to 12 Hz in the right brain hemisphere and (b) stimulating beta frequencies in a range of from 15 to 20 Hz in the left brain hemisphere for relieving symptoms of depression.

15. A photic and auditory stimulation method for pacing breathing in a subject to a predetermined breathing rate in the range of from 5 to 7 breath cycles per minute, the process comprising: (a) exposing the subject to an auditory cue; and (b) simultaneously exposing the subject to various stimulation frequencies or combinations of frequencies.

16. A method according to claim 15 wherein the auditory cue is a synthesized heartbeat sound.

17. A method according to claim 15 wherein the auditory cue is be provided at from two to four times the predetermined breathing rate.
